(19) United States
(12) Patent Application Publication
(10) Pub. No.: US 2003/0120341 A1
(43) Pub. Date: Jun. 26, 2003

(54) Devices and methods of repairing cardiac valves

(76) Inventors: Hani Shennib, Mt. Royal (CA); Albert Burdulis, San Francisco, CA (US)

Correspondence Address: BOZICEVIC, FIELD & FRANCIS LLP, 200 MIDDLEFIELD RD, SUITE 200, MENLO PARK, CA, 94025, US

(21) Appl. No.: 10/025,977

(22) Filed: Dec. 21, 2001

(51) Int. Cl. A61F 2/24

(52) U.S. Cl. 623/2.12; 623/904

(57) ABSTRACT

Devices and methods are provided for securing leaflets of a cardiac valve together. The subject devices include an assembly having a fastener, means for temporarily securing the fastener to the leaflets and means for permanently securing the fastener to the leaflets and means for anchoring the fastener to the heart wall. The subject methods are characterized by temporarily grasping the leaflets of a valve together at an apposition point, assessing at least one of blood flow and pressure gradient across said valve, determining whether to permanently secure the valve leaflets at said selected apposition point based upon at least one of the measured blood flow and pressure gradient, and performing one of permanently attaching the leaflets together at the apposition site or releasing the grasped leaflets. Also included are assemblies which include a subject device and a delivery device and may include a pressure monitoring member and/or a flow monitoring member. Kits which include the subject devices are also provided.

### FIELD OF THE INVENTION

The invention relates to devices and methods for the less invasive repair of cardiac valves, and particularly to less invasive repair of mitral and tricuspid valves.

### BACKGROUND OF THE INVENTION

The human heart has four valves; the aortic valve, the mitral valve, the pulmonary valve and the tricuspid valve. Various diseases and certain genetic defects of the heart valves can impair the proper functioning of the valves. Improper functioning of a valve can be severely debilitating and even fatal if left untreated, particularly if the diseased valve is the aortic valve (between the left ventricle and the aorta) or the mitral valve (between the left atrium and left ventricle). The common defects and diseases affecting each of these valves, and the treatments thereof, are typically different.

The aortic valve and, infrequently, the pulmonary valve, are prone to stenosis. Stenosis typically involves the buildup of calcified material on the valve leaflets, causing them to thicken and impairing their ability to fully open to permit adequate forward blood flow. Because stenotic damaged sustained by leaflets is irreversible, the most conventional treatment for stenotic aortic and pulmonic valves is the removal and replacement of the diseased valve.

On the other hand, the mitral valve and, less frequently, the tricuspid valve, are more prone to deformation, such as dilation of the valve annulus, tearing of the chordae tendinae and leaflet prolapse, which results in valvular insufficiency wherein the valve does not close properly and allows for regurgitation or back flow from the left ventricle into the left atrium. Deformations in the structure or shape of the mitral or tricuspid valve are repairable. Thus, because prosthetic valves have certain disadvantages that can have serious effects (e.g., mechanical valves carry the risk of thromboembolism and require anticoagulation treatment, and biological valves have limited durability), an improper functioning mitral or tricuspid valve is ideally repaired rather than replaced.

The mitral valve includes two leaflets or cusps, called the anterior and posterior leaflets, which are encircled by a dense fibrous ring of tissue known as the annulus. The leaflets are of unequal size with the posterior leaflet having a wider attachment area to the annulus. The end of the lines at which the leaflets come together are called the commissures. The leaflets are held in place by the chordae or threads connected at the base by two papillary muscles which extend from the underside of the leaflets to the papillary muscles within the wall of the left ventricle. The annulus of a normal mitral valve is somewhat “D” shaped.

The tricuspid valve, also an atrioventricular valve, functions similarly to the mitral valve but has three leaflets rather than two. The three leaflets, referred to as the anterior, posterior, and septal leaflets, and are roughly triangular in shape. Like the mitral valve leaflets, the tricuspid valve leaflets are encircled by a fibrous annulus and are held in place by chordae connected to associated papillary muscles. The annulus of the tricuspid valve is more nearly circular than is the mitral valve. While the two valves function very similarly, the mitral valve is subject to significantly higher back pressure than is the tricuspid valve and, as such, the mitral valve is more susceptible to degradation and deformation.

During systolic contraction of the heart, the free margins of the mitral leaflets and tricuspid leaflets, respectively, come in apposition to each other and close the respective atrial-ventricular passage. The chordae and papillary muscles hold the leaflets in this position throughout the systole cycle to prevent the leaflets from bulging into and opening within the associated atrium. However, when the valve or its leaflets are misshapen or enlarged, for example, when the annulus is dilated, the edges of the leaflets fail to meet each other, leaving an opening there between. This opening may involve lateral separation of the valve leaflets and/or elevation of one valve leaflet with respect to the other. In either case, the ineffective closure of the valve during ventricular contraction results in regurgitation or leakage of blood back into the atrium during ventricular contraction, and ultimately in reduced pumping efficiency. To compensate for such inefficiency in the mitral valve, for example, the left ventricle must work harder to maintain the requisite cardiac output. Overtime, this compensatory mechanism typically results in hypertrophy of the heart followed by dilation, i.e., an enlarged heart, which can lead to congestive heart failure.

Any one or combination of the annulus, the leaflets, the chordae and the papillary muscles may be the cause of the mitral and/or tricuspid insufficiency and/or regurgitation. Common conditions or diseases to the mitral and tricuspid valves which may result in mitral regurgitation include dilation of the annulus, ischemic regurgitation and myxomatous degeneration of the valve leaflet. Annular dilation typically involves the elongation or dilation of the posterior two-thirds of the mitral valve annulus, the section corresponding to the posterior leaflet. Ischemic regurgitation involves a lack of blood supply to the valve tissue, particularly the papillary muscles, due to coronary artery disease. Myxomatous degeneration involves weakness in the leaflet structure, leading to thinning of the tissue and loss of copation.

Various surgical techniques may be used to repair diseased or damaged mitral and tricuspid valves. These include but are not limited to annuloplasty (i.e., contracting the valve annulus to restore the proper size and shape of the valve), quadrangular resection of the leaflets (i.e., removing tissue from enlarged or misshapen leaflets), commissurotomy (i.e., cutting the valve commissures to separate the valve leaflets), shortening and transposition of the chordae tendonae, reattachment of severed chordae tendonae or papillary muscle tissue, and decalcification of valve and annulus tissue.

Another repair technique, commonly referred to as “bow-tie” repair, involves the edge-to-edge suturing together of the anterior and posterior leaflets. Typically, at least one suture is placed centrally with respect to the commissure line, creating a double orifice valve, thereby preventing prolapse at the central portions of the leaflets and reducing or eliminating regurgitation. The sutures may alternatively or additionally be placed closer to the commissures. These steps are typically performed using arrested, open heart techniques. Following the valve repair procedure, ultrasound is typically used to verify the repair.

Because they are performed on stopped hearts through an open chest approach, conventional valve repair techniques may require minimal instrumentation and time. However, because the success of the repair can only be tested on a beating heart, the heart must be closed up and the patient taken off the heart lung machine before testing can be done. If the repair is determined to be inadequate, the patient must be put back on cardiopulmonary bypass and the heart must be reopened.

Moreover, the risks and complications associated with open-heart surgery, which involves the use of cardiopulmonary bypass, aortic cross-clamping and cardioplegia arrest, are well known. The most serious risks of cardiopulmonary bypass and aortic cross-clamping are the increase in the likelihood of bleeding and stroke. Also, patients who undergo surgeries using cardiopulmonary bypass often require extended hospital stays and experience lengthy recoveries. Thus, while conventional heart valve surgery produces beneficial results for many patients, numerous others who might benefit from such surgery are unable or unwilling to undergo the trauma and risks of conventional procedures.

Within recent years, minimally invasive types of procedures for coronary artery bypass surgery have been developed which do not require stopping the patient's heart and the use of cardiopulmonary bypass; however, no such minimally invasive surgical procedure has been developed for the repair of cardiac valves.

Thus, it is desirable to provide a device which, when operatively used, involves a simplified procedure by which to repair a cardiac valve, in particular, mitral and tricuspid valves. For example, it would be beneficial to provide a device which, when properly implanted, corrects a defective valve in addition to other co-morbidities affecting proper function of the valve, obviating the need to perform ancillary procedures to correct leaflet size and shape, to adequately coapt the leaflets, to reattach or shorten chordae, etc. In addition, it is desirable to provide a valve repair procedure which requires minimal instrumentation and steps, is easier to perform than conventional valve repair procedures and reduces the time and cost of the procedure. Moreover, it is desirable to provide a valve repair procedure that obviates the need for cardiopulmonary bypass, can be performed on a beating heart, involves endovascular or less invasive techniques, can be performed on a patient while awake and/or in an ambulatory setting by surgeons, cardiologists or interventionalists.

### SUMMARY OF THE INVENTION

The present invention includes devices, methods and kits for repairing cardiac valves, particularly mitral and tricuspid valves experiencing regurgitation. The subject devices provide leaflet grasping and fastening functions, preferably performed by a single mechanism. The grasping function is used to apposition the valve leaflets such that the pressure gradient between the atrium and ventricle is optimized. The fastening function is used to permanently secure the leaflets together at least one location along their edges, i.e., along the commissure line. More specifically, the subject devices include an implantable fastener or clip having opposing jaws for grasping and temporarily and/or permanently fastening or holding opposing leaflet edges together at a selected point or points along the commissure line. The subject fastening or clip devices may be made of biodegradable or non-biodegradable materials as well as those materials which are inert and non-thrombogenic.

The implantable fastener or clip may be provided as part of an assembly for delivering, positioning and fastening or implanting the fastener or clip. The subject assembly may further include one or more means for evaluating or verifying the effectiveness of the one or more selected points of apposition prior to permanent placement of the fastener. Such evaluating or verification means may include pressure monitoring probes or components for measuring the pressures just above and just below the valve leaflets, i.e., in the atrium and the ventricle, respectively, and for determining the pressure gradient or differential there between. Additionally or alternatively, one or more flow monitoring probes may be included for measuring the normal flow and back flow of blood through the valve. The subject devices may further include a means for anchoring the fastener to appropriate location on the cardiac anatomy to prevent embolization of the fastener in case the fastener becomes unattached from the valve leaflets.

The subject fastening or clip devices may be configured for less invasive surgical and endovascular approaches, wherein the implantable clip or fastener and associated delivery, positioning, implanting and evaluation assembly are provided as part of a cannula or catheter assembly, respectively. As such, the implantable devices, flow probes and/or pressure monitors are configured for delivery through a cannula or catheter, or are themselves part of a cannula or catheter assembly.

The subject methods generally include delivering an implantable fastener or clip to the regurgitating valve to be repaired; monitoring the blood flow characteristics and/or pressure gradient at the valve; grasping together the valve leaflets at a selected point along the commissure line; determining, from monitoring the flow and/or pressure gradient characteristics, whether grasping at such selected point improves or optimizes the flow characteristics and/or pressure gradient, i.e., reduces regurgitation through the valve; and fastening the valve leaflets at one or more selected points wherein the flow/pressure are improved or optimized. The subject methods may further include anchoring the fastener to an appropriate location of the cardiac anatomy in order to prevent embolization of the fastener in case it becomes unattached from the valve leaflets.

Such methods may further include repeating the steps of grasping the leaflets, monitoring the blood flow characteristics and/or pressure gradient and determining whether the flow/pressure characteristics for each grasping step results in improvement or optimization in such flow/pressure characteristics. The above described steps of grasping and assessing flow and/or pressure may be repeated until one or more suitable apposition points are found, at which point(s) a fastener is locked into place onto the valve leaflets. As such, such methods further include the step of releasing the valve leaflets after the step of grasping the valve leaflets, upon a determination that there is no or insufficient improvement. Alternatively, the leaflets may be successively grasped (with or without subsequent release) and fastened together at more than one selected location, i.e., two or more of the subject fasteners are permanently attached to the valve leaflets, until sufficient improvement in flow and or pressure characteristics are achieved.

Thus, a feature of the present invention is that subject fasteners can be releasably or temporarily closed to grasp and secure the valve leaflets at a selected apposition point, but can also be re-opened or spread apart to release the leaflets if the apposition point is determined not suitable, and then subsequently reused.

The subject kits include at least one of the subject devices and/or assemblies for carrying out the subject methods.

These and other features and advantages of the invention will become apparent to those persons skilled in the art upon reading the details of the subject devices and methods as more fully described below.

### BRIEF DESCRIPTION OF THE DRAWINGS

The following drawings are provided and referred to throughout the following description, wherein like reference numbers refer to like components throughout the drawings:

FIG. 1A is a top view of an insufficient or defective mitral valve having leaflets which do not coapt with each other, resulting in regurgitation of blood from the ventricle into the atrium.

FIG. 1B is a cross-sectional view of the left side of the human heart, including the defective or insufficient mitral valve illustrated in FIG. 1A.

FIG. 2 is a perspective view of an embodiment of an implantable fastener and associated delivery and grasping assembly of the present invention.

FIG. 2A is a top view of the yoke of the assembly of FIG. 2.

FIG. 2B is side view of ajaw of the subject fastener of FIG. 2.

FIG. 3A shows the distal end of one embodiment of a fastener delivery device of the present invention configured to accommodate pressure and/or flow monitoring probes.

FIG. 3B is a top view of the delivery device of FIG. 3A.

FIG. 4A is a top view of the mitral valve of FIG. 1A, wherein the valve leaflets have been fastened at a selected apposition point along the commissure line.

FIG. 4B is a cross-sectional view of the left side of the human heart illustrating the result of the mitral valve of FIG. 4A having leaflets which have been fastened according to the methods of the present invention.

FIG. 5 illustrates an embodiment of a fastener of the present invention having an anchoring mechanism of the present invention.

FIG. 6 is a cross-sectional view of the left side of the human heart having mitral valve leaflets fastened with a fastener and attached anchoring mechanism of the present invention.

### DETAILED DESCRIPTION OF THE INVENTION

As mentioned above, the present invention includes devices, methods and kits for repairing cardiac valves, particularly mitral and tricuspid valves experiencing regurgitation.

Before the present invention is described in detail, it is to be understood that this invention is not limited to particular embodiments and applications described, as such may, of course, vary. For example, the following description of the invention is primarily described in the context of mitral valve repair; however, such description, with certain obvious modifications to the invention, is also intended to apply to the repair of tricuspid valves. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

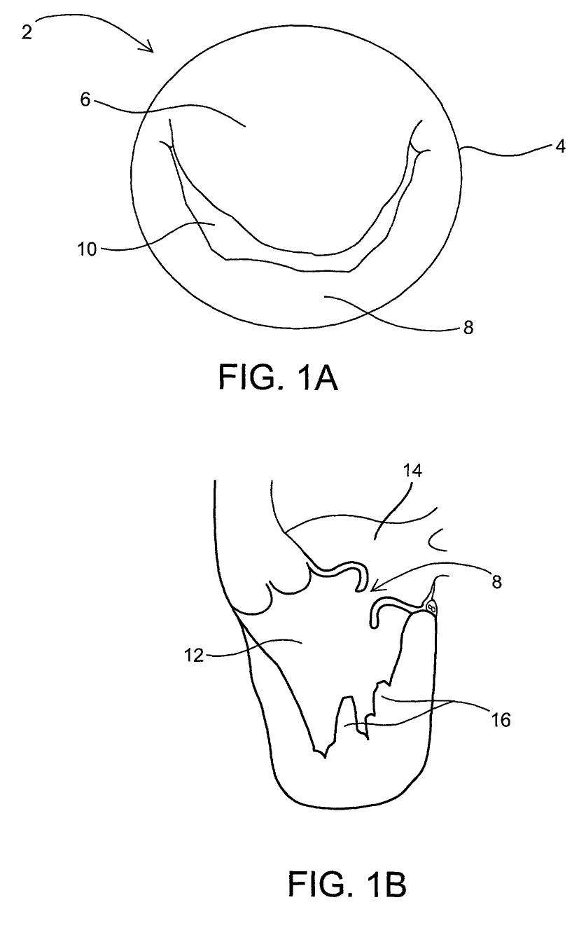

To better understand the present invention, FIGS. 1A and 1B are provided which illustrate a defective mitral valve 2 and the resulting effect on the functioning of the valve 2. Specifically, FIG. 1A illustrates a defective mitral valve 2 having an annulus 4, an anterior leaflet or cusp 6 and a posterior leaflet or cusp 8. Mitral valve 2 suffers from valvular insufficiency as evidenced by the gap 10 between the two leaflet edges during systole. FIG. 1B is a cross-sectional view of the left side of a heart having a left ventricle 12, a left atrium 14 and defective mitral valve 2 situated at the atrioventricular passageway there between. Mitral valve 2 is tethered to papillary muscles 16 by bundles of chordae tendinae (not shown). FIG. 1B further illustrates the effect that the dilation of mitral valve 2 has on its ability to properly function. Gap 10 may involve lateral separation of the valve leaflets and/or elevation of one valve leaflet with respect to the other. In all cases, the ineffective closure of the valve during ventricular contraction results in regurgitation or leakage of blood back into the atrium, thereby reducing the pumping efficiency of the heart during systole, i.e., reducing the amount of available oxygenated blood that is pumped by the left ventricle through the aortic valve to the body and brain.

The various embodiments of the devices of the present invention, which will now be described in detail, function to correct or improve the function of such a defective mitral valve 2. In further describing the present invention, devices of the present invention will be described first, followed by a description of the methods of using the subject device to temporarily or permanently fasten or clip leaflets of a valve together. Kits which include the subject devices will then be described.

Devices of the Present Invention

As mentioned above, the subject devices include an assembly that is capable of grasping and fastening leaflets of a defective valve at one or more apposition points along their edges, i.e., along the commissure line, either temporarily or permanently. The subject assemblies also include delivery means such as a sheath, e.g., a delivery catheter or cannula, and means for simultaneously monitoring certain, relevant cardiac characteristics such as cardiac pressure and/or flow to assess whether the fastening of the valve leaflets at the particular apposition point improves or optimizes blood flow and/or pressure, i.e., reduces regurgitation. The subject devices can be used to repair a variety of cardiac valves, wherein mitral valve repair applications will be used herein for exemplary purposes only, and is no way intended to limit the scope of the invention.

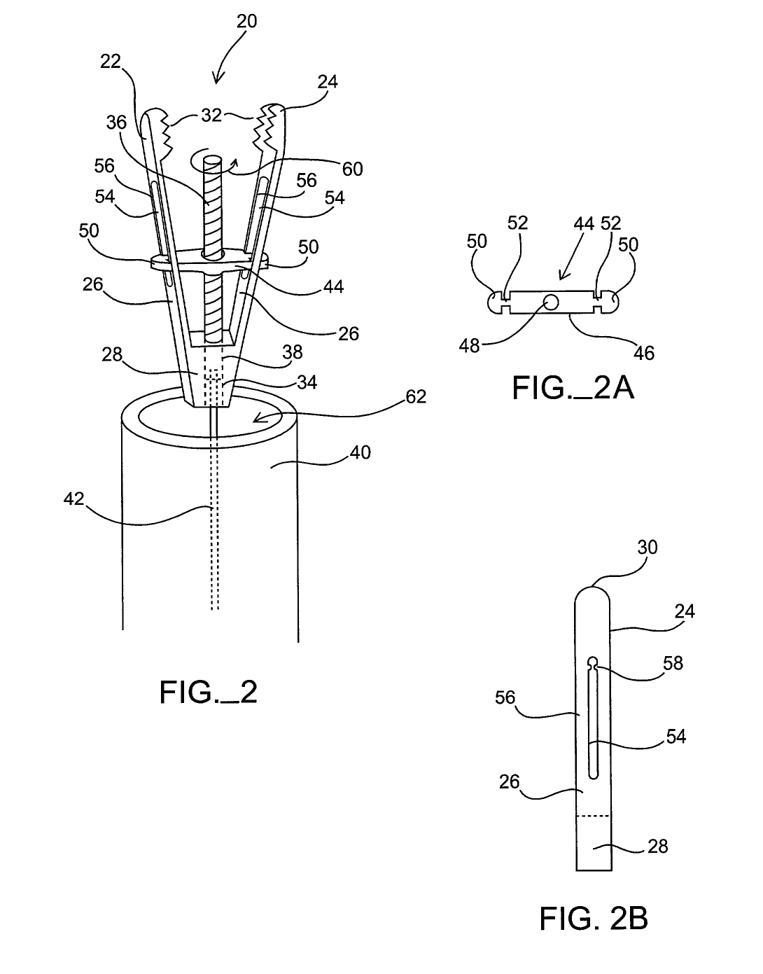

Referring now to FIG. 2, there is illustrated an exemplary embodiment of a device assembly 20 of the present invention. Assembly 20 includes an implantable fastener or clip 22 operatively associated with a delivery sheath 40. Fastener or clip 22 includes a jaw 24 having opposing jaw arms 26 which extend distally from a base portion 28. Jaw arms 26 have serrations or teeth 32 located on their inside or opposing distal surfaces for firmly but atraumatically grasping tissue there between. In this embodiment, teeth 30 are not designed to penetrate tissue grasped by jaw 24, but other embodiments of the subject devices may provide tissue-penetrating teeth. The distal ends 30 of jaws arms 26 are preferably rounded to avoid trauma to tissue it may come in contact with.

In their normally, biased open condition, jaw arms 26 define an acute angle sufficient to fit about the leaflets, and typically will be within the range from about 5° to 110° or more, and more typically within the range from about 30° to 60°. Jaw arms 26 may alternatively be configured to be biased in a closed position, wherein the above angle ranges would apply to their unbiased open positions. Jaw arms 26 and have lengths generally in the range from about 10 to 30 mm, but may be shorter or longer depending on the application and the size of the target heart valve being repaired. In order for jaws 24 to be positionable about or straddle the edges of the leaflets in an open position and to provide a sufficient grasping force in a closed position, the separation distance between distal end 30 of jaw arms 26 is generally in the range from about 6 to 10 mm but may be shorter or longer depending on the application and the size of the target heart valve.

Jaw arms 26 and base portion 28 may be formed of a unitary piece of a material that is substantially rigid, but nonetheless provides some flexibility such that jaw arms 26 will not break when operatively compressed together to grasp valve leaflets with a suitable gripping force. Alternatively, jaw arms 26 may be hinged to base portion 28 and spring-biased outward, where in this embodiment the jaw arms are also formed of a material that is substantially rigid, but nonetheless provides some flexibility such that jaw arms 26 will not break when operatively compressed together to grasp valve leaflets. In such a hinged configuration, the material of the jaw arms 26 and the base portion 28 to which it is hinged may be made from the same or different material, but usually the same material.

Regardless of whether the jaw arms 26 and the base portion 28 are formed of a unitary piece of material or whether they are separate, but hinged pieces, fastener 22 is made of any suitable biocompatible material. Such biocompatible materials may be permanently implantable, i.e., not biodegradable. Representative permanently implantable materials include, but are not limited to, plastics such as RC-1008 plastic, commonly used by those skilled in the medical device arts, and metals or alloys thereof such as titanium, stainless steel, aluminum, Nitinol and the like.

Fastener 22 may alternatively be made partially or wholly from bioresorbable or biodegradable materials such that fastener 22 becomes absorbed or degrades at a rate that is sufficient to allow the angiogenic and arteriogenic processes to form tissue adhesion between the leaflets Suitable biodegradable materials for fabricating fastener 22 include, but are not limited to, polyurethane, poly (L-lactic acid), polycaprolactone, poly (lactide-co-glycolide), poly (hydroxybutyrate), poly (hydroxybutyrate-co-valerate), polydoxanone, polyorthoester, polyanhydride, poly (glycololic acid), poly (D, L-lactic acid), poly (glycololic acid-co-trimethylene carbonate), polyphosphoester, polyphosphoester urethane, poly (amino acids), cyanoacrylates, poly (trimethylene carbonate, poly (iminocarbonate), copoly (ether esthers) (e.g., PEO/PLA) polyalkylene oxalates, polyphosphazenes, as well as biomolecules such as fibrin, fibrinogen, cellulose, starch, collagen and hyaluronic acid.

The fasteners may also have the ability to diffuse drugs or other agents at a controllable rate at the valve leaflet coaptation or apposition site. One or more therapeutic agents may be added to the base material during fabrication of the fastener and/or a coating containing such therapeutic agents may be applied to the fastener after it has been fabricated. Suitable therapeutic agents for use with the subject fasteners include, but are not limited to, dexamethasone, tocopherol, dexamethasone phosphate, aspirin, heparin, coumadin, urokinase, streptokinase and TPA, or any other suitable thrombolytic substance to prevent thrombosis at or around the apposition point between the valve leaflets. Such therapeutic agents may be applied by spraying, dipping or other means. The subject fasteners may also be seeded with endothelial cells to promote angiogenesis between the fastener and the valve leaflet. Still further, the subject fasteners may include materials such as paralyne or other hydrophilic substrates that are biologically inert and reduce surface friction, where such materials may be applied by spraying, dipping or any other convenient means.

Furthermore, the fastener may be configured to enable fluoroscopic visualization while delivering and operatively placing the fasteners on the valve leaflets. Fastener 22 may comprise one or more radio-opaque materials added to the fastener's base material during the fabrication process or a coating containing radio-opaque material may be applied to the fastener after it has been fabricated. Alternatively, fastener 22 may be provided with one or more radiopaque markers. Any suitable material capable of imparting radio-opacity may be used, including, but not limited to, barium sulfate, bismuth trioxide, iodine, iodide, titanium oxide, zirconium oxide, metals such as gold, platinum, silver, tantalum, niobium, stainless steel, and combinations thereof.

Base portion 28 has a threaded thru-hole 34 along the central longitudinal axis of clip 22. The proximal end 38 of an alignment pin 36 is in threaded engagement within thru-hole 34. Threaded about alignment pin 36 is a yoke 44, best illustrated in FIG. 2A. Yoke 44 has an elongated body portion 46 having a threaded center thru-bore 48. The opposing end portions 50 of yoke 44 each have a necked-downed or keyed portion 52 configured to matingly engage with corresponding ways or grooves 54, best illustrated in FIG. 2B, which extend lengthwise through the central portion of jaw arms 26, respectively.

A drive rod 42 is provided which is releasably attachable to the proximal end 38 of alignment pin 36. When in operative engagement with alignment pin 36, drive rod 42 extends proximally from threaded thru-hole 34 through delivery sheath 40 and preferably beyond the proximal end (not shown) of delivery sheath 40. At its proximal end (not shown), drive rod 42 is provided with means, e.g., a handle or a power-operated mechanism, for rotating or turning rod 40 in clockwise and counter-clockwise directions about its longitudinal axis. The rotation of drive rod 42 in turn rotates alignment pin 36 in a corresponding direction along its axis, such as the direction designated by arrow 60 of FIG. 2. Such rotation causes yoke 44 to translate along the longitudinal axis of alignment pin 36. As such, end portions 50 of yoke 44 are caused to translate within grooves 54 along the respective longitudinal axes of jaw arms 26. Rotation in one direction causes yoke 44 to translate distally or upwards towards jaw 24, and rotation in the opposite direction causes yoke 44 to translate proximally or downward towards base portion 28 of fastener 22. As yoke 44 moves distally, jaw arms 26 are caused to draw closer together until yoke 44 reaches the distal end of each groove 54 wherein respective detents 58 are provided for locking yoke 44 permanently in place in such distal-most position, i.e., yoke 44 may not be then be translated in the reverse or proximal or downward direction. As such, jaw arms 26 are permanently closed and locked in place. Drive rod 42 may then be rotated in the opposite direction, thereby unthreading and detaching itself from pin 36. Prior to permanently locking yoke 44 within detents 58, however, yoke 44 may be selectively translated proximally or distally along alignment pin 36. Proximal translation of yoke 44 causes jaw arms 26 to move apart from each other.

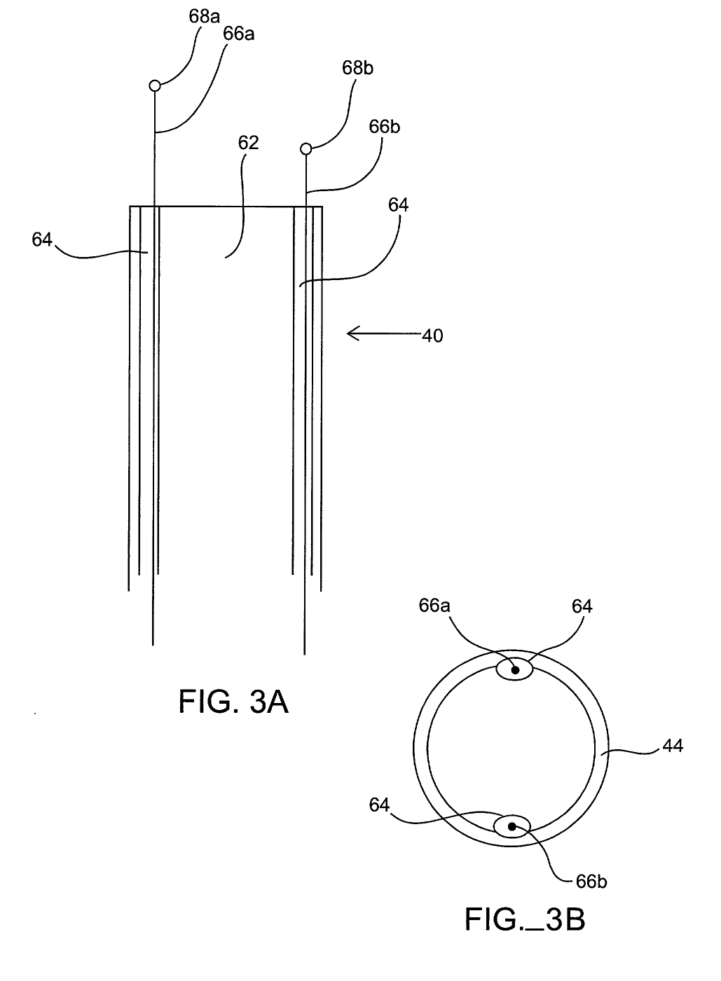

As described above, device assembly 20 includes a delivery sheath 40 for delivering fastener 22 to the appropriate area of the heart, i.e., delivering the fastener 22 to the area of the defective valve leaflets. Generally, sheath 40 has a proximal end, a distal end and at least one lumen there between. FIGS. 3A and 3B show an exemplary embodiment of a delivery sheath 40 according to the subject invention. Delivery sheath 40 defines a lumen 62 and usually has a tubular configuration. The dimensions and material of such sheath 40 depend on the size of fastener 22 and the type of approach or access route a physician employs to access the target cardiac valve to be repaired.

For an endovascular approach, and cardiac valve applications in particular, a catheter is used as the delivery sheath 40. Catheters suitable for accommodating the fasteners of the present invention include those sized generally from about 6 to 30 French, but may be smaller or larger depending on the application and the intended delivery path to the target heart valve. Such catheters have lengths generally in the range from about 100 to 300 cm, but may be shorter or longer depending on the application and the intended delivery path to the target heart valve. As will be explained in further detail below, it is preferable that the internal diameter of catheter 40, i.e., the diameter of lumen 63, be no greater than the maximum separation distance between jaw arms 26. If for practical reasons a larger diameter catheter 40 is to be used, a secondary sheath or catheter (not shown) having a lumen diameter which meets such requirement and is deliverable within lumen 62 and over rod 42 may be employed.

Materials suitable for use in the subject delivery catheters are chosen to provide the desired catheter flexibility and rigidity in order to manipulate the catheter through a patient's vasculature. The materials used to manufacture the catheter may also include radio-opaque materials, where such radio-opaque materials may include, but are not limited to, barium sulfate, bismuth trioxide, iodine, iodide, titanium oxide, zirconium oxide, gold, platinum, silver, tantalum, niobium, stainless steel, and combinations thereof.

In many embodiments of the subject catheters, the catheters are steerable so that the clinician may temporarily impart a desired curve to the catheter from a remote location in order to be navigated within the patient's anatomy, e.g., through the patient's cardiovascular system. As such, drive rod 42 may have a flexible configuration to accommodate and further facilitate such steerability. A variety of steering mechanisms known to those of skill in the art may be employed to impart the desired steerability. Generally, steerable catheters includes one or more pull wires which extend through the catheter shaft, and connect to the catheter adjacent the distal end of the catheter at an off-axis location. The pull wires connect to a control knob or knobs, slide actuator, or other suitable manipulating member that is mounted in a control handle. Representative catheters suitable for use with the subject invention include, but are not limited to, those used for electrophysiology, which are well known in the art.

For direct but less invasive or endoscopic approaches where the subject devices are delivered through a trocar port placed in the body, e.g., in the chest cavity, and delivered endoscopically to the target location, delivery sheath 40 is preferably a cannula. For cardiac valve applications, cannula 40 typically has a diameter in the range from about 4 to 12 mm, and more typically from about 6 to 8 mm, and lengths typically in the range from about 10 to 30 cm, and more typically from about 15 to 25 cm. As mentioned above with respect to the catheter-type sheaths of the present invention, if the lumen or internal diameter of cannula 40 is greater than that of the maximum separation distance between jaw arms 26, a secondary sheath (not shown), such as another cannula or a catheter, having a smaller diameter lumen may be employed which has an internal diameter not greater than the maximum separation distance between jaw arms 26.

In either endovascular or endoscopic approaches, the catheter and cannula delivery devices of the present invention may further include additional lumens 64, as illustrated in FIGS. 3A and 3B, for delivering ancillary instrumentation for facilitating the implantation of the subject fasteners and clips. For example, these additional lumens 64 may be used to deliver pressure and/or flow monitoring probes 66a, 66b to the target valve to be repaired. The monitoring element 68a of one probe 66a may be delivered to one side of the valve, e.g., within the right atrium to measure blood pressure just above the mitral valve, and a second monitoring element of 68b of the second probe 66b may be delivered to the other side of the valve, e.g., within the left ventricle to measure blood pressure just below the mitral valve. Alternately, a single probe having two spaced-apart monitoring elements may be used. With this alternate embodiment, the probe is delivered to a point where the distal monitoring element is positioned on the side of the valve opposite the delivery device and the proximal monitoring element is positioned on the side of the valve proximate the delivery device. With either embodiment, a pressure monitoring system (not shown) of the type known in the art external to the patient then measures the difference between the two pressures on opposing sides of the valve leaflets. Similarly, as mentioned above, a monitoring element may be positioned just above the mitral valve to measure back flow, if any, during systole. A variety of pressure monitoring probes and flow monitoring probes, which are known in the art, may be used with the subject invention. Additionally, other instrumentation, such as guide wires, endoscopes, and secondary grasping devices, may be delivered through additional lumens 64.

Methods of the Present Invention

Also included in the present invention are methods for repairing cardiac valves, e.g., mitral valves. In the subject methods, leaflets of a heart valve are brought together and temporarily grasped at a first apposition point along their edges, i.e., along the commissure line. Once temporarily grasped, the suitability of securing the leaflets together at the particular coaptation or apposition point is assessed by measuring one or more relevant characteristics related to the heart while the valve leaflets are grasped together, such as blood flow and/or pressure, to verify the effectiveness of fastening the leaflets together at this apposition point. If the flow and/or pressure characteristics are not improved or are insufficient, the leaflets are released and then grasped again at another apposition point, where such an apposition point is similarly evaluated for suitability. Once an apposition point is determined suitable, the valve leaflets are permanently fastened at that apposition point along the commissure line. The subject steps may be repeated to successively grasp (with or without subsequent release) and fasten together the leaflets at more than one selected apposition point along the commissure line until sufficient improvement in flow and/or pressure is achieved.

Accordingly, the first steps in the subject methods is to gain access to the area of heart which includes the valve to be repaired and then to advance a subject fastener to the site. As mentioned above, an endovascular approach may be used which includes navigating a sheath such as a catheter through the vasculature of the patient and delivering a valve repair device there through, where the position of the catheter may be continuously verified by fluoroscopy and/or by transesophageal echocardiogram. Alternatively, a more direct approach may be used wherein the heart is accessed through a trocar port placed in the body, e.g., in the chest cavity and delivering a valve repair device through a sheath such as a cannula positioned through the port. Furthermore, while it is possible to perform the valve repair procedures described herein on a stopped heart, the procedures described herein are preferably performed on a beating heart, which will allow certain characteristics such as blood flow and/or pressure to be assessed during the procedure and eliminate the risks associated with cardiopulmonary bypass.

In those embodiments employing an endovascular or percutaneous approach to mitral valve repair using a sheath such as a catheter to access the heart, there exists two procedures which may be used: a retrograde approach and a transeptal approach. In the transeptal approach, the catheter is introduced into a patient's body percutaneously by means of a modified Seldinger technique via the right femoral vein. By means of transesophegeal echocardiogram, the catheter is then visualized, guided and advanced into the inferior vena cava and into the right atrium of the heart. The catheter then crosses the atrial septum through a small atrial septostomy (created by cardiological techniques known in the art) to enter the left atrium of the heart. For example, a guide wire may be placed across the atrial septostomy and the catheter may then be threaded along the guide wire into the left atrium. The distal end or working end of the catheter can then be placed or brought to rest at a predetermined position in, at, or in proximity to the mitral valve. When performing the subject methods to repair a tricuspid valve, there is no need for a transeptal approach. Instead, an approach from the right atrium into the right ventricle may be employed. In a retrograde endovascular approach, a catheter is introduced into a patient's body via a femoral artery. By means of transesophogeal echocardiogram visualization and guidance, the catheter is then advanced into the aorta, crossing the aortic valve into the left ventricle and the distal end or working end of the catheter can then be placed or brought to rest at a position in, at, or in proximity to the mitral valve, preferably at the underside of the mitral valve.

In those embodiments employing a direct access approach, the heart may be accessed by means of a traditional surgical approach, e.g., through a sternotomy, a thoracotomy, or a sub-xyphoid approach, or through one or more endoscopic ports positioned with in the chest cavity, e.g., between adjacent ribs. Once access to the heart is achieved, an entry site within a wall of the heart or a great vessel is created. More specifically, a penetrating means such as a trocar, obturator or guide wire or the like is used to penetrate the myocardium. If entry through the left ventricle or right ventricle is preferred for repair of the mitral valve and tricuspid valve, respectively, the apex of the heart is a suitable location to penetrate due to its resiliency to trauma. On the other hand, the entry site may be made in the wall of the left atrium or right atrium, respectively.

A fastener delivery sheath 40 can then be inserted through the opening in the heart and brought to a position in, at, or in proximity to the mitral valve, preferably the underside of the mitral valve leaflets. Visualization and guidance of sheath 40 may be accomplished by transesophageal echocardiogram. Once delivery sheath 40, such as the catheters or cannulas described above, is distally advanced and properly positioned in, at, or in proximity to the mitral valve, the blood flow and/or pressure gradient across the valve may be measured (although not required to be) such as by means of the pressure/flow monitoring devices described above, where such measurements may be used as baseline reference measurements. In other words, these measurements, i.e., one or both of pressure and flow measurements, may be made prior to grasping the valve leaflets so as to determine the base line or reference measurement of the blood flow and/or pressure gradient of the defective valve. Another set of measurement may be then be made after the valves have been grasped. The second measurement or sets of measurements, i.e., post-leaflet grasping measurements, may then be compared to the first measurement or sets of measurements, i.e., pre-leaflet grasping, base line, or reference measurements, to determine the efficacy, i.e., the improvement on valve function, e.g., the reduction in regurgitation during systole, of attaching the valve leaflets at the selected apposition point. Such comparison, i.e., the determination of the change in the pre- and post-leaflet grasping measurements is performed by a flow and/or pressure monitoring and control device, such as a microprocessor, operatively coupled to the proximal end of the one or more flow and/or pressure probes which extend proximally outside the patient's body.

In those embodiments of the subject methods where baseline measurements are performed before the valve leaflets are grasped, delivery sheath 40 is positioned adjacent either just above or below the leaflets of the valve to be repaired and flow and/or pressure monitoring probes are advanced out of the delivery catheter, i.e., out of one or more additional lumens of the delivery device to the target valve to be repaired. For example, a first pressure monitoring element may be advanced to one side of the valve and a second pressure monitoring element may be advanced to the other side of valve to measure the pressure on both sides of the valve during systole, i.e., the pressure differential or gradient across the valve may be measured during contraction of the heart. As described above, the pressure monitoring elements may be from a single probe, e.g., a single probe having spaced apart monitoring elements, or may be from different probes. In addition to or in place of the above described pressure measurement, a flow measuring element may also be advanced to the site of the target valve. More specifically, the flow probe is advanced out of the delivery device and positioned within the left atrium just above the valve leaflets and flow is measured during systole. As mentioned above, these measurements may be used as baseline or reference measurements against which to compare flow and/or pressure measurements taken after the leaflets have been brought together at one or more apposition points along their edges; however these pre-leaflet grasping measurements, i.e., the baseline measurements, may not and/or need not be performed in every instance.

Once baseline measurements are obtained, or in the case where baseline measurements are not first obtained, a subject valve leaflet fastener or clip 22, as described above, is then advanced through delivery sheath 40 to the valve to be repaired, e.g., the mitral valve. More specifically, the subject fastener or clip 22 is selectively positioned with respect to the valve leaflets by distal advancement of delivery sheath 40 and/or of drive rod 42 within the lumen of delivery sheath 40. Usually, whether employing an endovascular, endoscopic or direct approach, fastener 22 is delivered through the left ventricle to the underside of the regurgitating mitral valve or, in the case of the tricuspid valve, through the right ventricle to the underside of the regurgitating tricuspid valve. A point of apposition, i.e., a desired fastening point, between the leaflets is then selected. Fastener 22 is then positioned such that the valve leaflets are positioned between opposing teeth 32 of jaw arms 26. Preferably, this step is performed during a systolic cycle as the leaflet cusps or edges are closer together and, thus, easier to grasp. Sheath 40, or a secondary sheath as discussed above, is then advanced distally over jaw arms 26, causing jaw arms 26 to move together and thus temporarily grasp the leaflet tissue there between.

When in a closed position, either temporarily or permanently, jaw arms 26 maintain the valve leaflets secured between them by exerting a gripping force on the leaflets to sufficient to secure them between teeth 32. In many embodiments, teeth 32 do not penetrate the jaw leaflets. However, in certain embodiments of the subject invention, the teeth 32 include sharp tips which can penetrate the leaflets.

Once the edges of the valve leaflets are temporarily grasped together between teeth 32, the effectiveness of securing the leaflets at the selected apposition point may be evaluated by measuring the blood flow and/or pressure gradient during systole while the valve leaflets are temporarily grasped. Similar to the steps for measuring the baseline flow and/or pressure, a first pressure monitoring element may be advanced to one side of the temporarily grasped valve and a second pressure monitoring element may be advanced to the other side of the temporarily grasped valve to measure the pressure on both sides of the valve leaflets. As mentioned above, in addition to or in place of the above-described pressure measuring assessment, flow may be measured and assessed. As such, a flow monitoring element may be positioned above the grasped mitral valve, i.e., in the left atrium of the heart, and blood flow may be measured during systole. Such flow/pressure measurements may then be assessed by comparison to the baseline measurements, if obtained previously, and/or assessed independently based on therapeutically or clinically relevant criteria or standards known to those skilled in the art.

Based on these assessments, it is determined whether or not fastener 22 should be removed from or permanently fastened at the selected apposition point. If it is determined that the flow and/or pressure is not improved or optimized by securing the valve leaflets at the selected apposition point, the delivery sheath 40 is moved proximally, causing jaw arms 26 to open and release the grasp on the valve leaflets. Another apposition point is then selected and the above-described steps for temporarily grasping and assessing the particular apposition point is repeated until an apposition site is determined to be suitable based on the assessment of blood flow and/or pressure with the subject fastener temporarily secured. The same fastener 22 may be used as described above at these one or more successive selected points of apposition until fastener 22 is permanently fastened to the valve leaflets.

If, however, it is determined that the pressure and/or flow is improved or optimized by grasping the valve leaflets at the selected apposition point, the subject fastener 22 may then be permanently attached to the valve leaflets at the apposition point. While maintaining jaw arms 26 in a closed position by means of sheath 40, drive rod 42 is rotated in the direction which will rotate pin 36 and distally translate yoke 44 until yoke 44 is positioned within detents 58. As such, jaw arms 26 are permanently closed and fastener 22 is permanently fastened to the valve leaflets. Drive rod 42 is then rotated in the opposite direction to release fastener 22, including jaw 24 and pin 36, permanently secured to the valve leaflets at the selected apposition point.

In addition to using transesophageal echocardiogram techniques to visualizing and guiding the delivery sheaths 40 and fasteners 22 of the present invention, transesophageal echocardiogram may be employed in the subject methods to perform the steps of determining pre- (i.e., baseline) and post-grasping flow characteristics of the valve. The physician would then compare the pre- and post-grasping flow characteristics to assess the resulting improvement, if any, of placing a subject fastener at the selected coaptation or apposition site(s).

The physician may choose to terminate the procedure upon permanent placement of this first fastener, or elect to permanently place one or more additional fasteners according to the above procedures. If the physician elects to terminate the procedure, the delivery device and the flexible rod 42 are removed from the heart and, ultimately, the body cavity. If, however, the physician elects to permanently place one or more additional fasteners, the subject methods also include “re-loading” delivery sheath 40 with an additional or subsequent subject fastener and placing the additional fastener according to the above-described procedures. More specifically, after permanently placing the first or previous fastener, the delivery device may remain in place in the vicinity of the valve while drive rod 42 is removed from the body cavity through delivery device sheath 40. A second or subsequent fastener 22 may then be threaded onto the same drive rod 42 or otherwise provided attached to another drive rod 42, and advanced through delivery sheath 40 to the defective valve. The steps described above are then repeated as appropriate.

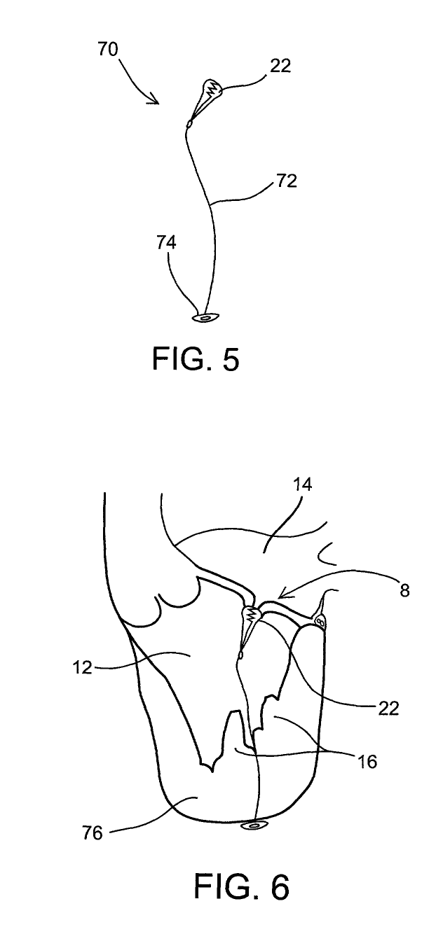

After a subject fastener 22 has been permanently attached to the valve leaflets, fastener 22 may be further anchored or secured to the heart in order to minimize or eliminate the risk of embolizing fastener 22 should it some how become unattached from the valve leaflets. More specifically, as shown in FIG. 5, an anchoring mechanism 70 having an anchor line 73 in the form of a fiber, wire or suture, may be attached at one end to fastener 22 and at the other end to an anchor 74, having, for example, a clip or button configuration. As shown in FIG. 6, anchor 74 is configured to be placed or penetrated into or through the ventricle wall 77 or otherwise attached to a papillary muscle of the heart (not shown).

If for some reason fastener 22 were to become dislodged from the leaflets, either intraoperatively or postoperatively, the anchoring mechanism would prevent fastener 22 from traveling beyond the left ventricle. In the endovascular methods of the present invention, delivery sheath or catheter 40 is configured to retain anchoring mechanism 70 during delivery and placement of a fastener 22. After permanent placement of a fastener 22, catheter 40 is steered and manipulated to release anchor line 72 and to fix anchor 74 to an appropriate location within the heart wall. In the direct access methods of the present invention, cannula 40 is configured such that it retains anchoring mechanism 70 and, after permanent placement of a fastener 22 to the leaflets of the target valve, cannula 40 is pulled for removal through the cannula entry site within the heart wall. Upon exiting the cannula entry site, anchoring line 72 is pulled there through, and thereafter cannula 40 or other means delivered through cannula 40 is used to fix anchor 74 to the heart wall. Suitable anchoring locations on the heart wall are within or on the outside of the ventricle wall 76, or within a papillary muscle. In either of the above methods, anchor 74 may be alternatively fixed to the cardiac anatomy prior to grasping the leaflets.

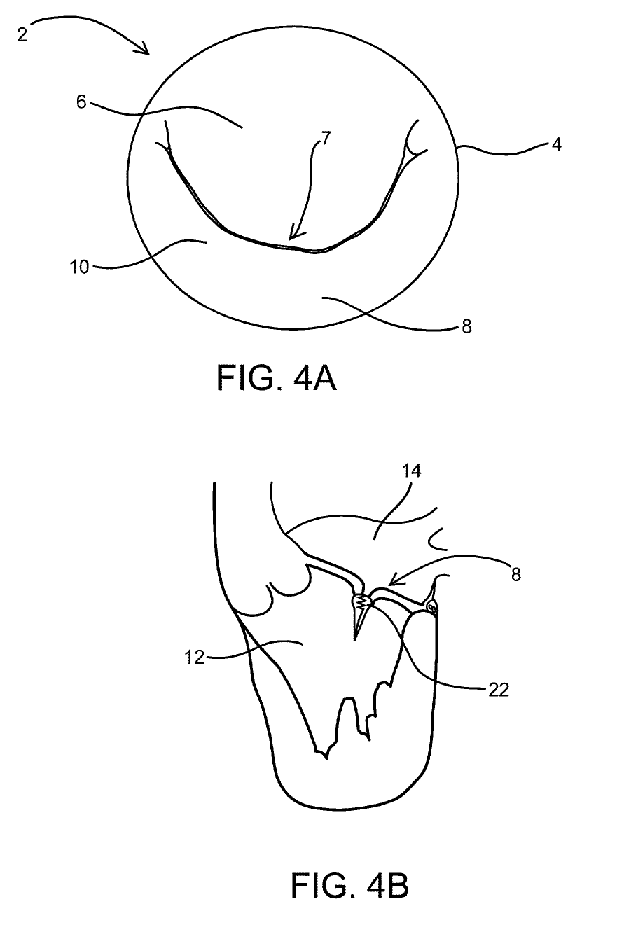

FIG. 4A shows mitral valve 2 of FIG. 1A after it has been repaired according to the subject methods and with the subject fasteners. FIG. 4A is a top view of mitral valve 2 repaired, thus having an anterior leaflet or cusp 6 and a posterior leaflet or cusp 8 attached at a selected apposition point 7 along the commissure line. As shown in FIG. 4A, the gaping commissure line 10 present during systole as shown in FIG. 1A is no longer present. FIG. 4B is a cross-sectional view of the left side of the human heart showing leaflets of repaired mitral valve 2 of FIG. 4A secured together at apposition point 7 with a subject fastener 22.

The subject methods may further include the absorption or degradation of the subject fastener at a rate that is sufficient to allow the angiogenic and arteriogenic processes to form tissue adhesion between the leaflets. In other words, the fastener 22 may break down after a set time period, during which time the apposition point of the leaflets is reinforced with vascularized tissue in-growth producing a sufficiently strong bond between the valve leaflets. Furthermore, one or more therapeutically relevant drugs or agents, discussed above, may be delivered or diffused to the defective valve and more specifically to the fastened apposition points, where such delivery or diffusion at a controlled rate by any convenient means discussed above.

Kits of the Present Invention

Also provided by the subject invention are kits for use in practicing the subject methods. The kits of the subject invention at least include a subject fastener or subject assembly, as described above. The subject kits may also include a plurality of such subject fasteners or assemblies. The subject fasteners may be provided with an anchoring mechanism, as described above. The subject kits may further include one or more flow monitoring probes and/or one or more pressure monitoring probes. Furthermore, the subject kits may include additional instrumentation for performing the subject methods, where such additional instrumentation may include, but is not limited to, one or more guide wires, trocars, guide catheters, etc. Finally, the kits may further include instructions for using the subject fasteners and/or assemblies for repairing cardiac valves. The instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., CD-ROM, diskette, etc.

It is evident from the above description and discussion that the above described invention provides a device which, when operatively used, involves a simplified procedure by which to a repair cardiac valve, and, in particular, mitral and tricuspid valves. The above described invention provides a number of advantages, including the ability to temporarily grasp the valve leaflets and perform blood flow and/or pressure measurements while the leaflets are temporarily grasped to verify whether grasping the leaflets at the particular point improves or optimizes flow and/or pressure before permanently fastening the leaflets together. The subject invention also effectively corrects a defective valve in addition to other co-morbidities affecting proper function of the valve, obviating the need to perform ancillary procedures to correct leaflet size and shape, to reattach or shorten chordae, etc. Furthermore, the subject methods require minimal instrumentation and steps, is easier than conventional valve repair procedures to perform and reduces the time and cost of the procedure. As such, the subject invention represents a significant contribution to the art.

All publications and patents cited in this specification are herein incorporated by reference as if each individual publication or patent were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. An assembly for securing leaflets of a cardiac valve together at a point of apposition between the valve leaflets, said assembly comprising:
a fastener;
a means for temporarily securing said fastener to said leaflets; and
a means for permanently securing said fastener to said leaflets.

2. The assembly according to claim 1, further comprising a delivery sheath for delivering said fastener from outside a patient's body to said valve leaflets.

3. The assembly according to claim 2, wherein said sheath is selected from the group consisting of a catheter and a cannula.

4. The assembly according to claim 1, wherein said assembly further includes at least one of a pressure monitoring probe and a flow monitoring probe.

5. The assembly according to claim 4, wherein at least one of said pressure monitoring probe and said flow monitoring probe is configured to be delivered through a lumen of said delivery sheath.

6. The assembly according to claim 2, wherein said delivery sheath comprises said means for temporarily securing said fastener to said leaflets.

7. The assembly according to claim 2 wherein said fastener comprises said means for permanently securing said fastener to said leaflets.

8. The assembly according to claim 1 further comprising an anchoring mechanism attached to said fastener.

9. A method for securing together leaflets of a cardiac valve of a heart having an apex, said method comprising:
(a) temporarily grasping the leaflets of a valve together at a selected apposition point;
(b) measuring at least one of blood flow and pressure gradient across said valve;
(c) determining whether to permanently secure said valve leaflets at said selected apposition point based upon at least one of said measured blood flow and pressure gradient; and
(d) performing one of permanently securing said leaflets together at said selected apposition site or releasing said grasped leaflets.

10. The method according to claim 9, further comprising, prior to said step (a), measuring one of at least blood flow and pressure gradient across said valve to obtain a baseline measurement(s).

11. The method according to claim 10, wherein step (c) comprises comparing said measurement(s) of step (b) with said baseline measurement(s).

12. The method according to claim 9, further comprising repeating said steps (a) through (d).

13. The method according to claim 12, wherein said steps (a) through (d) are repeated until the measurement(s) of step (b) indicates that the functioning of said valve leaflets is sufficiently improved.

14. The method according to claim 9 wherein said method is performed using the assembly of claim 1.

15. The method according to claim 9 wherein said method is performed by means of an endovascular approach.

16. The method according to claim 9 wherein said method is performed by means of a surgical approach.

17. The method according to claim 16 further comprising accessing said cardiac valve through an entry site formed within the apex of the heart.

18. The method according to claim 9 wherein said method is performed while the heart is beating.

19. A method for repairing a regurgitating cardiac valve having at least two opposing leaflets, said method comprising:
(a) providing the assembly of claim 1;
(b) delivering said fastener to said leaflets;
(c) selecting a point of apposition between said leaflets;
(d) temporarily causing said fastener to grasp said leaflets at said selected point of apposition; and
(e) assessing at least one of blood flow and pressure gradient across said leaflets; and
(f) determining whether to permanently secure said fastener to said leaflets at said selected apposition point based upon at least one of said assessed blood flow and pressure gradient.

20. The method according to claim 19, upon determining not to permanently secure said fastener to said leaflets at said selected apposition point, further comprising:
(g) causing said fastener to release said grasped leaflets;
(h) selecting a second point of apposition between said leaflets;
(i) repeating steps (d), (e) and (f).

21. The method according to claim 19, further comprising:
(g) permanently securing said leaflets together at said selected apposition site.

22. The method according to claim 21, further comprising:
(h) repeating steps (a) through (f) for one or more additional selected apposition sites.

23. The method according to claim 21, further comprising:
(h) anchoring said fastener to a location on the cardiac anatomy.

24. The method according to claim 23, wherein said fastener is anchored to the ventricle wall.

25. The method according to claim 19, wherein said steps (b), (c) and (d) are performed with the assistance of transesophageal echocardiogram.

26. The method according to claim 19, wherein said blood flow is assessed by means of transesophageal echocardiogram.

27. A kit for repairing a cardiac valve, said kit comprising;
an assembly according to claim 1; and
a plurality of said fasteners.

28. The kit according to claim 27 further comprising a fastener delivery sheath configured for endovascularly delivering said fastener to said cardiac valve.

29. The kit according to claim 27 further comprising a fastener delivery sheath configured for delivering said fastener to said cardiac valve through a surgical opening within the chest cavity of a patient.
